(19) United States
(12) Patent Application Publication
(10) Pub. No.: US 2009/0132012 A1
(43) Pub. Date: May 21, 2009

(54) Method for pretreating patient before surgery

(75) Inventor: Steven C. Shanks, McKinney, TX (US)

Correspondence Address: ETHERTON LAW GROUP, LLC, 5555 E. VAN BUREN STREET, SUITE 100, PHOENIX, AZ, 85008, US

(73) Assignee: Therapy Products, Inc.

(21) Appl. No.: 11/985,828

(22) Filed: Nov. 16, 2007

(51) Int. Cl. A61B 18/20

(52) U.S. Cl. 607/89

(57) ABSTRACT

This invention is a method of pre-treating a patient with low-level laser energy to reduce pain and swelling caused by subsequent injury, thereby speeding recuperation. The preferred embodiment applies low-level laser energy at about 635 nm prior to surgery. In a preferred embodiment prior to breast augmentation, 635 nm laser energy emitted from a laser source of less than 1 W is applied for about 3 minutes to the patient's breast prior to breast augmentation surgery. In an alternate embodiment, the same low-level laser energy is applied for about 4 minutes to the patient's breast after breast augmentation surgery, as well. The method is applicable to pre-disease states which are expected to be followed by injury.

### FIELD OF INVENTION

This invention relates generally to methods for improving recuperation after surgery or other intentional injury. More particularly, this invention relates to a method of pre-treating a patient with low-level laser energy prior to a disease state, to reduce the pain and swelling caused by the subsequent injury and thereby speed recuperation.

### BACKGROUND

Low energy laser therapy (LLLT) is used in the treatment of a broad range of conditions. LLLT improves wound healing, reduces edema, and relieves pain of various etiologies, including successful application post-operatively to liposuction to reduce inflammation and pain. It is also used in the treatment and repair of injured muscles and tendons. LLT has also been used to treat hearing loss, hair loss, acne and other skin disorders.

The common thread of the low-level laser treatments known to date is that they have been for the treatment of disease states of varying degree. That is, the laser energy is applied after the patient is injured via surgery, trauma, disease or other mechanism. It would be desirable to treat a body prior to injury to reduce pain and swelling caused by the subsequent injury and thereby improve recuperation.

Although counterintuitive, injury is often voluntarily and intentionally undertaken with the expectations of subsequent benefits. For example, breast augmentation is intentional injury for expected improvement in physical shape; some facial cosmetic procedures such as dermabrasion intentionally damage the facial tissue so that fresh cells will replace the damaged ones; and facelifts severely injure a patient's face, with hopes of a younger appearance after the injury from the procedure heals. Even excessive exercise can sometimes lead to intentional injury, as an athlete works out so hard that his muscles break down, with the expectation of improved performance after the muscles heal. It would be desirable to reduce the discomfort of intentional injury and speed post-injury recuperation.

### SUMMARY OF THE INVENTION

This invention is a method of pre-treating a patient with low-level laser energy to reduce pain and swelling caused by subsequent injury, thereby speeding recuperation. The preferred embodiment applies low-level laser energy at about 635 nm prior to surgery. In a preferred embodiment prior to breast augmentation, 635 nm laser energy emitted from a laser source of less than 1 W is applied for about 3 minutes to the patient's breast prior to breast augmentation surgery. In an alternate embodiment, the same low-level laser energy is also applied for about 4 minutes to the patient's breast after breast augmentation surgery, as well. The method is applicable in any pre-injury state which is expected to be followed by injury.

### BRIEF DESCRIPTION OF THE DRAWINGS

FIG. 1 is a side view of a patient being treated with low level laser energy from a hand-held probe before breast augmentation surgery.

FIG. 2 is a front view of a patient's breast being treated with a linear beam spot prior to breast augmentation.

### DETAILED DESCRIPTION OF THE INVENTION

The present invention is a method of treating typically healthy patients with low-level laser energy prior to an intentional injury. The laser energy applied is low level, that is, the treatment has a dose rate that causes no immediate detectable temperature rise of the treated tissue and no macroscopically visible changes in tissue structure. Consequently, the treated and surrounding tissue is not heated and is not damaged.

Breast augmentation surgery is used as the primary example herein, but the method can be applied prior to any intentional or otherwise expected injury. Breast augmentation surgery involves making an incision in or near the breast and inserting an implant either between the breast and the chest muscle (subglandular placement) or behind the chest muscle (submuscular placement). Submuscular placement may make surgery last longer, may make recovery longer, may be more painful, and may make it more difficult to have some re-operation procedures than the subglandular placement, but it may result in less noticeable implants, less capsular contracture, and easier imaging of the breast with mammography. The preferred embodiment of the method herein applies to either type of surgery.

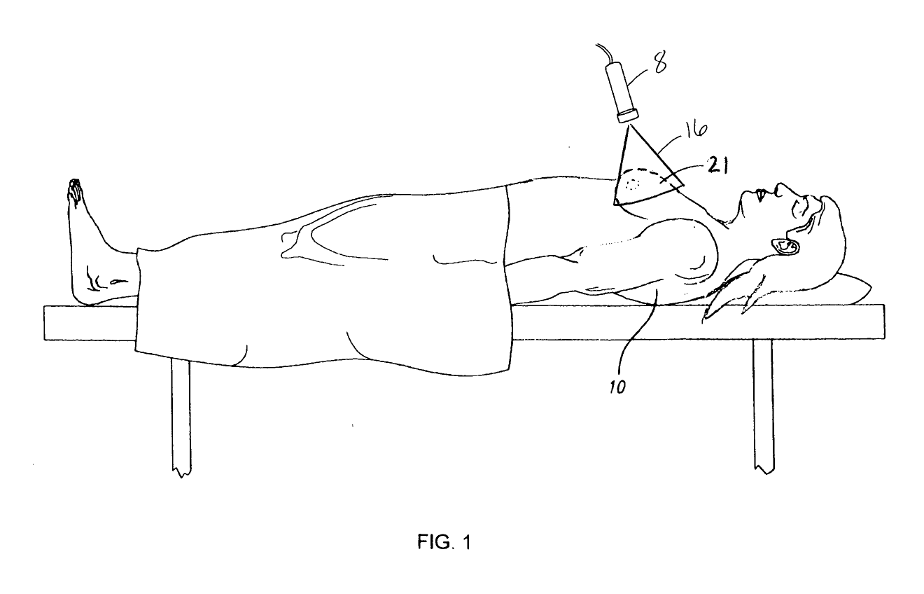

FIG. 1 illustrates the preferred embodiment in which, prior to breast augmentation surgery, a patient 10 is treated in the area generally encompassing the breast, referred to herein as the treatment zone 21. Preferably the laser energy 16 is applied externally through the skin of the patient by scanning the laser energy 16 across the treatment zone 21. Sufficient laser energy is applied to reduce the pain and edema resulting from such surgery. In general, the treatment comprises applying laser energy to the treatment zone for 1-12 minutes. Preferably, pulsed laser energy emitted from a 635 nm laser source of less than 1 W is applied for about 3 minutes to the patient's breast prior to breast augmentation surgery. In an alternate embodiment, the same low-level laser energy is also applied for about 4 minutes to the patient's breast after breast augmentation surgery. The duration, frequency, and laser energy characteristics will vary for each patient, depending on such factors as the type of expected injury, the age and thickness of the tissue being treated, and the composition of the tissue, e.g. fat content, etc.

Preferably the laser light is visible to the human eye so that the area of application is easily determined, generally using wavelengths of between about 400 nm-800 nm. A laser device that provides this low-level energy is known in the art as a cold laser, such as the invention described in U.S. Pat. No. 6,013,096. Other lasers known in the art for use in low-level laser therapy include Helium-Neon lasers having a 632 nm wavelength and semiconductor diode lasers with a broad range of wavelengths between 405-1500 nm. Low-level lasers are available commercially.

The preferred laser energy source 11 is a semiconductor diode emitting laser light. Semiconductor diode lasers known in the art for use in low-level laser therapy can emit a broad range of wavelengths between 400-800 nm. Other preferred laser energy sources known in the art for use in low-level laser therapy include Helium-Neon lasers having a 632 nm wavelength. The laser device may have one or more laser energy sources. In the preferred embodiment, the laser device has two semiconductor diodes emitting light substantially simultaneously, each at about 635 nm. Other embodiments are contemplated wherein the laser device contains a plurality of semiconductor diodes emitting different wavelengths, but one or more of the semiconductor diodes can be turned off so that the laser device emits light of only one color. In another embodiment, the laser device contains one or more semiconductor diodes of a single color. Different therapy regimens require diodes of different wattages. The preferred laser diodes use less than one watt of power each. In an alternative embodiment, the laser energy source is remotely located and the laser light is conducted by fiber optics to the treatment zone.

In a preferred embodiment, the treatment comprises applying red laser energy to the treatment zone. In an alternate embodiment, more than one wavelength of laser energy is applied to the treatment zone in each treatment. In one example, red and violet visible light is used, more preferably at about 635 nm and 405 nm, respectively. The red and violet light is preferably applied substantially simultaneously, but may also be applied alternately. In alternative embodiments, success may be had with the use of only red laser energy, only violet laser energy, or a combination of red and violet laser energy, depending on the patient.

The method may use various pulse frequencies of 0-100,000 Hz. The preferred embodiment changes pulse frequency about every 30 seconds from 4 Hz, to 12 Hz, to 28, Hz and to 16 Hz. A single pulse frequency may be used, or changing pulse frequencies may be used. The pulse frequencies may change in a random pattern or a predictable pattern, including patterns that are linear, saw tooth, stepped, sinusoidal, exponential, Gaussian, bell-shaped, or shaped otherwise.

The laser energy is preferably applied by a scanning laser, in either a hand-held probe 8 (shown in FIG. 1) or a stationary source that emits laser energy, such as an arm attached to a stand or a wall. A stationary source scanning laser is disclosed in U.S. patent application Ser. No. 10/976581 filed on Oct. 29, 2004, which is incorporated herein by reference. Alternatively, the laser energy can be applied by freely moving a non-scanning laser over the treatment zone 21. A doctor, nurse or other therapist may apply the laser energy, or the patient may do it herself.

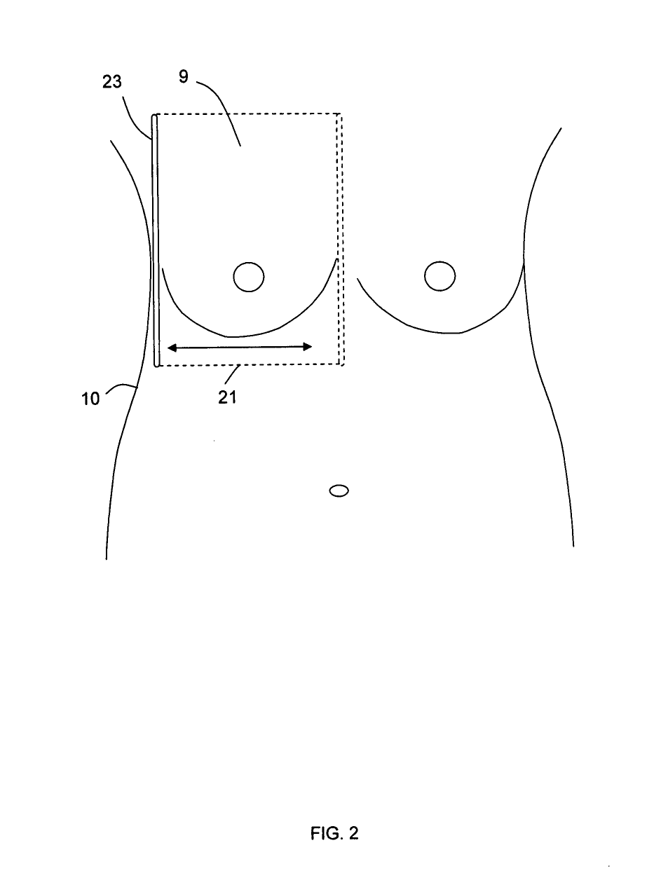

FIG. 2 illustrates the preferred embodiment of applying laser energy to a treatment zone 21 by moving a linear beam spot 23 generated by a scanning or non-scanning laser back and forth across the treatment zone 21. In another embodiment, a line of laser light is rotated rapidly to form a substantially circular treatment zone. A laser to accomplish such scanning is disclosed in U.S. patent application Ser. No. 11/409,408 filed on Apr. 20, 2006, which is incorporated herein by reference. In yet another embodiment, the laser energy is applied to a treatment zone 21 created on the patient's breast by scanning a circular beam spot rapidly across the desired area. A laser to accomplish such scanning is disclosed in U.S. Pat. No. 7,118,588. A shield may be employed to prevent the laser light from reflecting or deflecting to undesired locations.

While has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

I claim:

1. A method for pre-treating a patient prior to injury to improve post-injury recuperation, the method comprising:
a) applying low-level laser energy to the patient prior to injury.

2. The method of claim 1 wherein the injury is intentional.

3. The method of claim 1 wherein the injury is surgery.

4. The method of claim 1 wherein the injury is breast augmentation.

5. The method of claim 1 wherein the injury is dermabrasion.

6. The method of claim 1 wherein the injury is a facelift.

7. The method of claim 1 wherein the injury is muscle injury as a result of exertion.

8. The method of claim 1 wherein the application of laser energy reduces post-injury pain.

9. The method of claim 1 wherein the application of laser energy reduces post-injury swelling.

10. The method of claim 2 wherein the application of laser energy reduces post-injury time to heal.

11. The method of claim 1 wherein the laser energy is in the visible spectrum.

12. The method of claim 1 wherein the laser energy is red.

13. The method of claim 1 wherein the laser energy is 635 nm.

14. The method of claim 1 wherein the laser energy is a line of laser light.

15. A method for pre-treating a patient prior to breast augmentation surgery comprising:
a) applying laser energy to a breast of the patient prior to breast augmentation surgery for about 1-12 minutes;
b) wherein the wavelength of the laser energy is about 635 nm; and
c) wherein laser energy is provided by a laser source having power of less than 1 watt.

16. The method of claim 15 wherein the laser energy is applied for about 3-4 minutes.

17. The method of claim 15 further comprising:
a) applying the laser energy to the breast of the patient after breast augmentation surgery for about 1-10 minutes.

18. The method of claim 15 further comprising:
a) laser energy having a wavelength of about 405 nm.
